(19) United States
(12) Patent Application Publication
(10) Pub. No.: US 2011/0300116 A1
(43) Pub. Date: Dec. 8, 2011

(54) Method for Generating a Recombinant Clonal Cell Line and Novel Reagents for Use in the Method

(75) Inventors: Stephen John Hill, Nottingham (GB); Barrie Kellam, Nottingham (GB); Richard John Middleton, Nottingham (GB)

(73) Assignee: CELLAURA TECHNOLOGIES LTD, Nottingham (GB)

(21) Appl. No.: 13/133,366

(22) PCT Filed: Sep. 29, 2008
(86) PCT No.: PCT/GB08/03294

§ 371 Date: Jun. 7, 2011

(30) Foreign Application Priority Data
Sep. 28, 2007 (GB) 0718935.0

(51) Int. Cl. A61K 35/12; C07D 487/14; A61K 35/00; C07F 5/02; A61P 43/00; G01N 21/64; C07D 223/16

(52) U.S. Cl. 424/93.21; 435/7.2; 540/495; 540/523; 546/13; 548/110; 544/229; 424/93.2

(57) ABSTRACT

A method for generating a recombinant clonal cell line expressing a target cell surface receptor at a specific level of expression from a cell population comprising cells transfected with a plasmid encoding the cDNA sequence of the target receptor and expressing the target cell surface receptor, the method comprising (c) incubating the cell population with a receptor specific fluorescent ligand (d) selecting single cells from step (c) expressing the target cell surface receptor by monitoring the specific binding of the fluorescent ligand using flow cytometry; and novel fluorescent ligands.

This invention relates to a method for generating a recombinant clonal cell line and novel reagents for use in the method.

### BACKGROUND

A major requirement in the study of the pharmacology and signalling characteristics of cell surface receptors is the availability of recombinant cell lines expressing a particular transfected receptor at a specific expression level. These cell lines are also of immense value in the screening of compound libraries for new therapeutic agents. Traditionally, the generation of clonal cell lines involves the laborious process of: (a) transfection; (b) antibiotic resistant selection of cells expressing a particular receptor and finally (c) the dilution cloning (i.e. from single cells) of cells expressing a particular level of the cell surface receptor. The most labour intensive and time consuming aspect of this process is the dilution cloning and identification of clones expressing receptors at a particular level. This invariably requires all clonal lines to be expanded to the stage at which there are sufficient numbers of cells for radioligand binding to be performed. This usually requires in excess of a million cells.

In cells expressing a recombinant version of the receptor containing a green fluorescent protein (GFP) tag, this latter step is made easier by the ability to use fluorescence activated cell sorting (FACS) to identify cells expressing the receptor protein at a particular level.

The disadvantage of this approach, however, is that the GFP tag can alter the pharmacological characteristics of the cell surface receptor. There is therefore a need for a method which enables identification of single cells expressing the receptor protein at a specific level, prior to expansion of the cell line, without affecting the pharmacological characteristics of the cell surface receptor.

### BRIEF SUMMARY OF THE DISCLOSURE

In accordance with the present invention there is provided a method for generating a recombinant clonal cell line expressing a target cell surface receptor at a specific level of expression from a cell population comprising cells transfected with a plasmid encoding the cDNA sequence of the target receptor and expressing the target cell surface receptor, the method comprising

(c) incubating the cell population with a receptor specific fluorescent ligand
(d) selecting single cells from step (c) expressing the target cell surface receptor by monitoring the specific binding of the fluorescent ligand using flow cytometry.

Suitably step (c) provides for fluorescent labelling of the cell population with a receptor specific fluorescent ligand that binds to the target cell surface receptor.

Preferably single cells selected in step (d) are suitable for cloning into a clonal cell line. Preferably the method comprises e) expansion of selected single cells from step (d) into a clonal cell line.

Preferably the method comprises generating the cell population by means of

(a) transfecting cells with a plasmid encoding the cDNA sequence of the target receptor and an antibiotic selection marker; and
(b) antibiotic resistant selection of those cells expressing the particular cell surface receptor thereby generating a cell population.

Suitably therefore the invention comprises a method for generating a recombinant clonal cell line expressing a target cell surface receptor at a specific level of expression comprising:

(a) transfecting cells with a plasmid encoding the cDNA sequence of the target receptor and an antibiotic selection marker;
(b) antibiotic resistant selection of those cells expressing the particular cell surface receptor thereby generating a cell population
(c) incubating the cell population with a receptor specific fluorescent ligand
(d) selecting single cells from step (c) expressing the target cell surface receptor by monitoring the specific binding of the fluorescent ligand using flow cytometry; and
(e) expansion of selected cells into a clonal cell line.

Preferably flow cytometry comprises the use of a fluorescence activated cell sorter (FACS™) or fluorescence activated cell sorting technique.

In a further aspect the invention provides the use of a recombinant clonal cell line obtained by the method of the invention in the screening of compound libraries for new therapeutic agents, or in the study of pharmacology and signalling characteristics of cell surface receptors. Preferably the cell line comprises a trace amount of fluorescent ligand. Reference herein to a trace amount is to any residual amount remaining after clonal expansion of a single cell into a cell line. A trace amount of fluorescent ligand may be dissociated from a cell or may remain bound to a cell.

In a further aspect the invention provides a kit for use with the method comprising a set of instructions together with one or more fluorescent ligands for use in the method.

In a further aspect the invention provides the use of known and novel fluorescent ligands in the method.

In a further aspect the invention provides the use of a ligand identified by the method of the invention for drug targets selected from GPCRs, ligand-gated ion channels and tyrosine kinase receptors.

In a further aspect the invention provides novel fluorescent ligands.

In this invention, we have used long-acting fluorescent ligands that bind to a cell surface, such as to GPCRs to provide a reversible fluorescent tag for (FACS™) analysis and sorting that will allow cells expressing a particular level of GPCR to be identified at the single cell level and separated for further expansion in cell culture. Over time the fluorescent ligand will dissociate from the cells. Furthermore as selected cells are expanded into a cell colony, the majority of cells will not be labelled. The clonal cell line produced is therefore the wild type receptor. For example a ligand associated with the fluorophore BODIPY 630/650 via an appropriate spacer provides a molecule that is retained at the cell surface receptor long enough to allow FACS™ sorting.

### BRIEF DESCRIPTION OF THE DRAWINGS

Embodiments of the invention are further described hereinafter, by way of example, with reference to the accompanying drawings, in which:

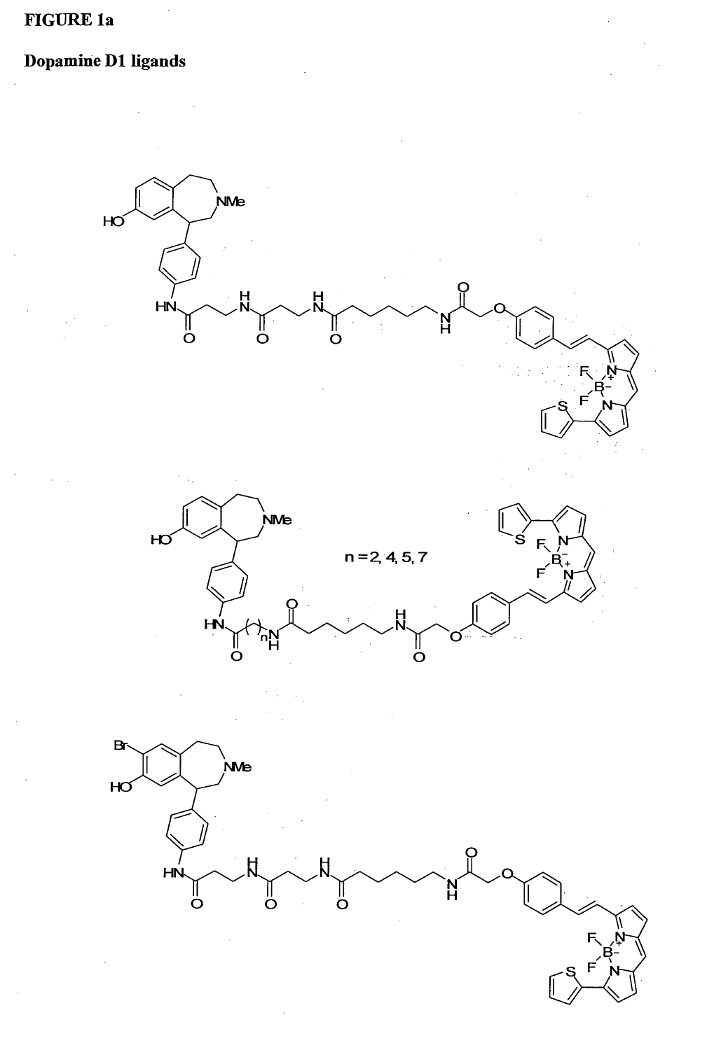

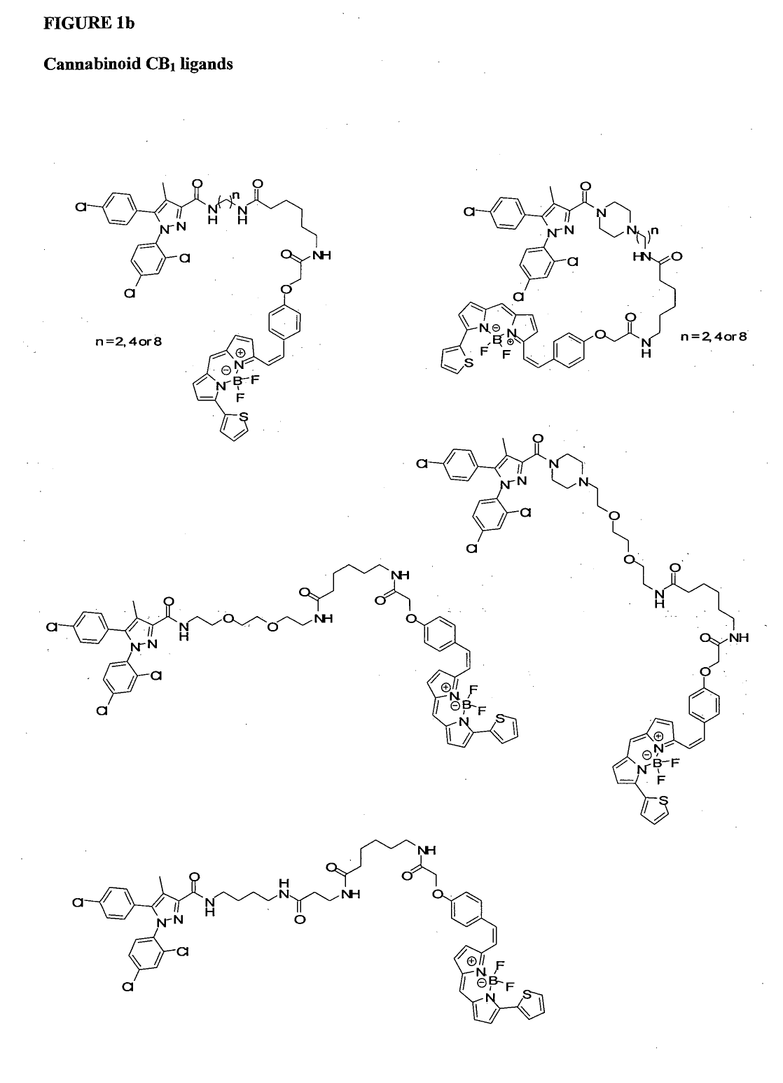

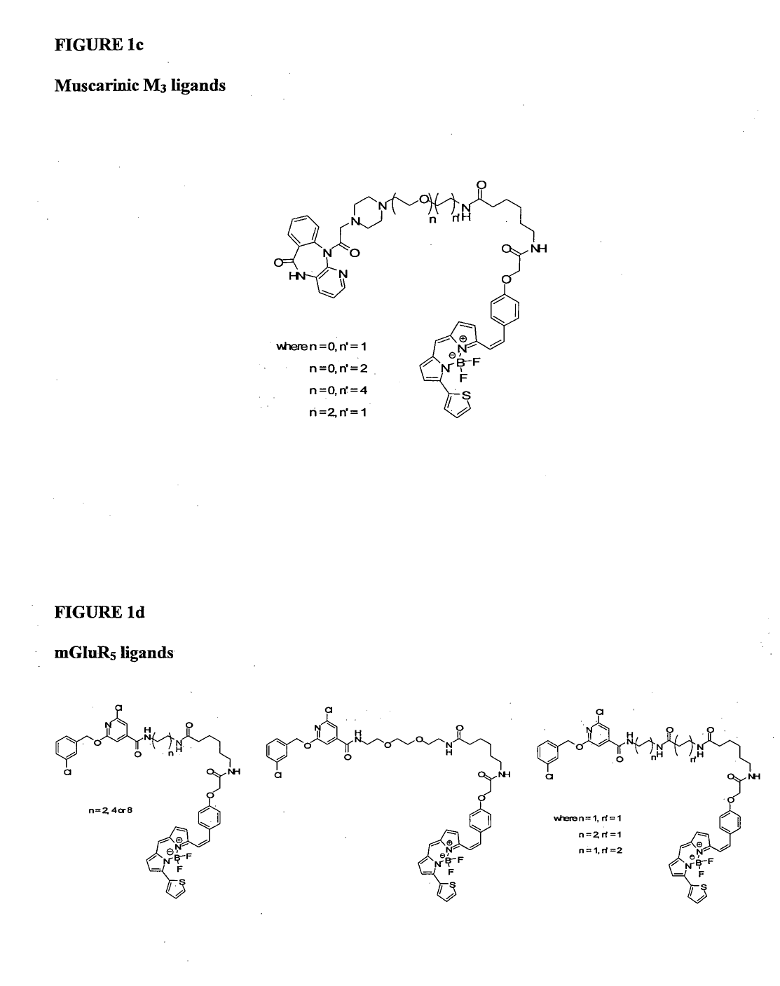

In FIG. 1 are novel fluorescent ligands of the invention for use in the method of the invention.

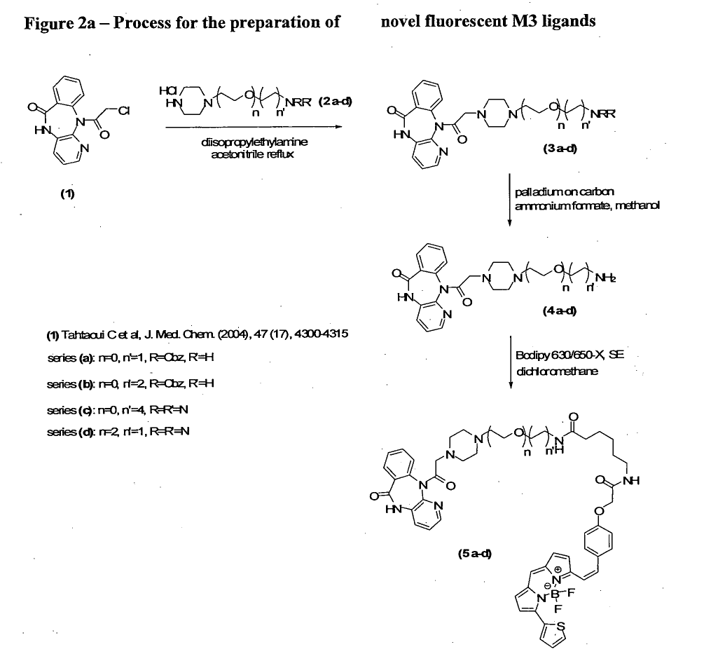

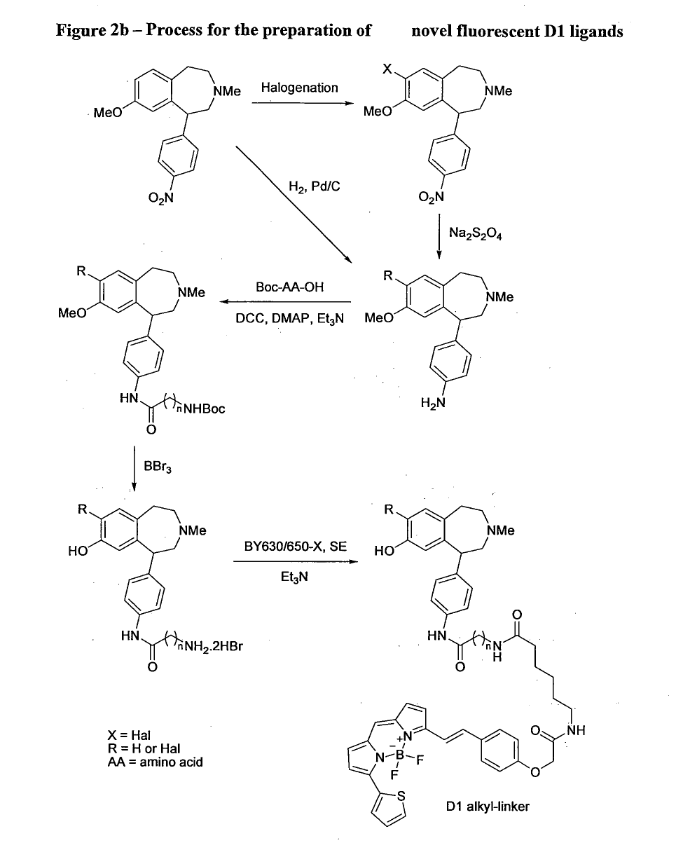

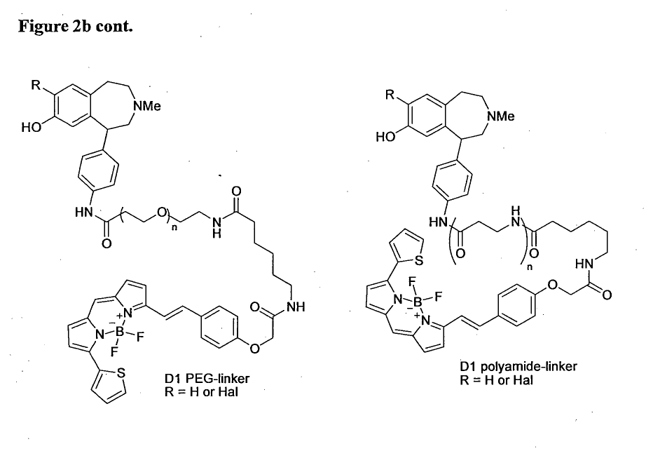

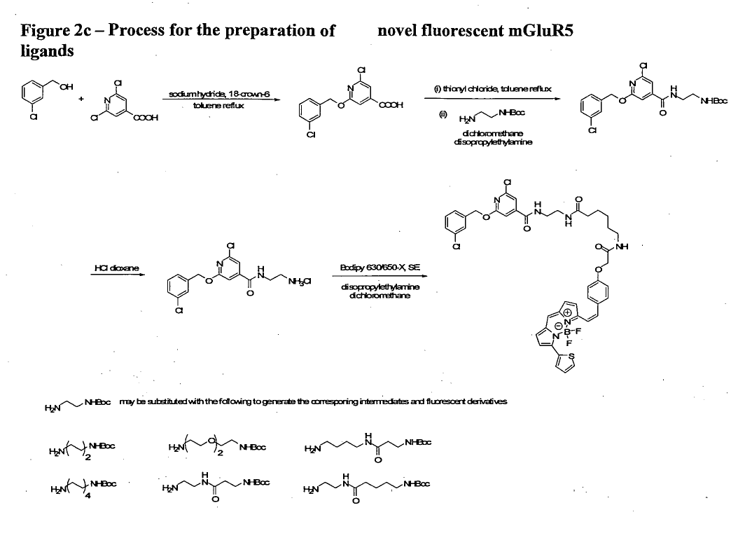

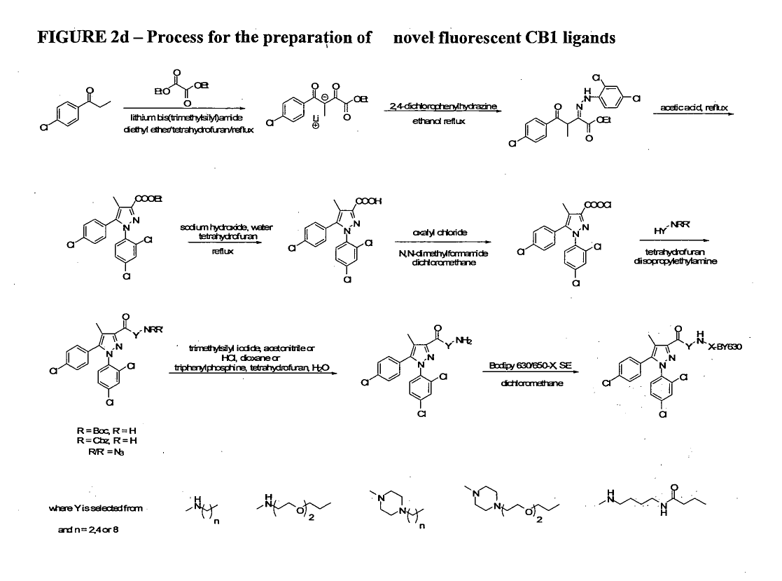

In FIG. 2 are illustrated processes for the preparation of novel fluorescent ligands of the invention. FIG. 2c is a general scheme that is applicable to the synthesis of D1 alkyl, PEG or polyamide linker compounds, which are prepared by substituting the relevant amino acid (Boc-AA-OH).

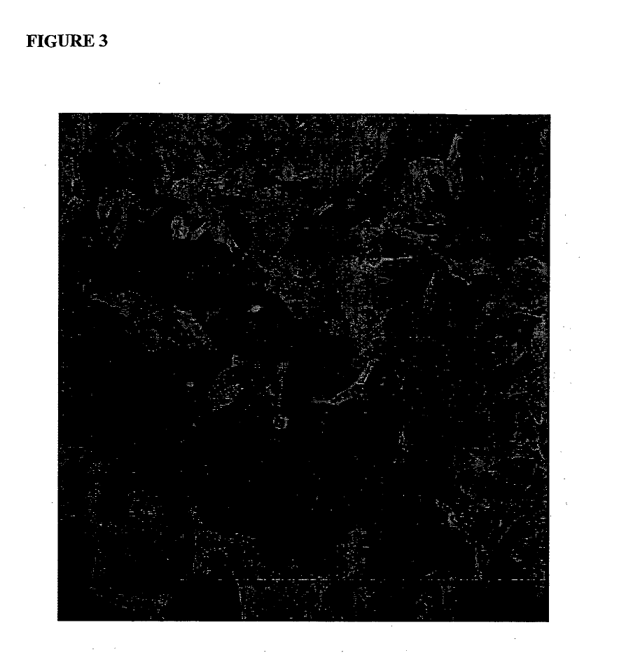

In FIG. 3 is shown a confocal microscope image of N-{4-aza-3,7-dioxo-7-[4-(8-methoxy-3-methyl-2,3,4,5-tetrahydro-1H-3-benzazepine-1-yl)phenylamino]heptyl}-6-{2-[4-(2-(4,4-difluoro-4,4a-dihydro-5-(thiophen-2-yl)-4-bora-3a,4a-diaza-s-indacene-3-yl)vinyl) ligand (10 nM) binding to human dopamine D1-receptor expressing CHO “cell line” before antibiotic resistance selection and prior to FACS™ sorting. Red images (light grey) show cells binding the fluorescent D1-receptor antagonist and the blue images (dark grey) show the Hoechst staining of the cellular nuclei. It is clear that only about 20% of the cells are expressing the human dopamine D1 receptor.

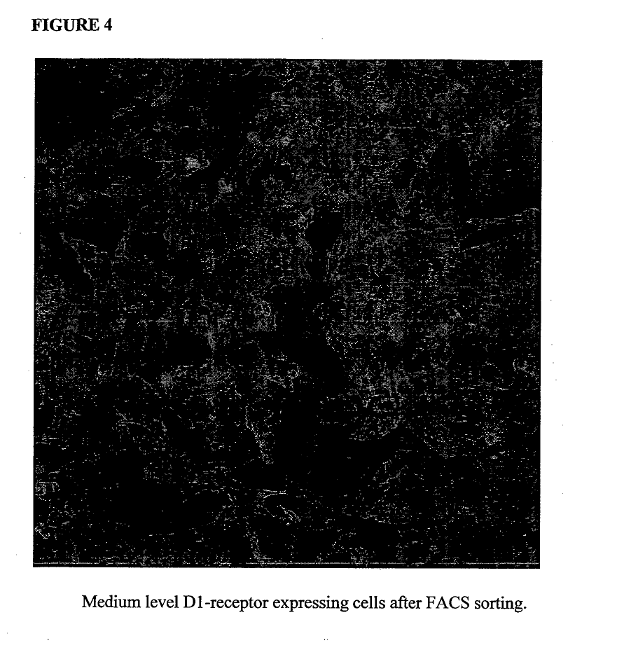

In FIG. 4 is shown a confocal microscope image of CHO cells expressing dopamine D1 receptors labelled with the fluorescent N-{4-aza-3,7-dioxo-7-[4-(8-methoxy-3-methyl-2,3,4,5-tetrahydro-1H-3-benzazepine-1-yl)phenylamino]heptyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide (red stain shown as light grey). Nuclei of cells were stained with Hoechst 33342 (blue stain shown as dark grey). Shown are medium level D1-receptor expressing cells after FACS sorting.

### DETAILED DESCRIPTION

Reference herein to flow cytometry is to the rapid sequential analysis of single cells, usually using laser light and fluorescent labels. This can be used to identify single cells with a particular fluorescence intensity (at a particular fluorescence wavelength) and these cells can then be sorted into populations with a particular level of fluorescence intensity or into single cells in a multiwell plate. Reference herein to a fluorescence activated cell sorter (FACS™) or fluorescence activated cell sorting technique is to an apparatus or technique which enables sorting a suspension of biological cells into two or more containers, one cell at a time, based upon specific light scattering and fluorescence characteristics of each cell.

Reference herein to a wild type cell line is to a cell line which is of naturally occurring type, and is naturally occurring or cloned from a naturally occurring cell.

Reference herein to cell culture is to a synthetic culture for the promotion of cell viability, growth and reproduction.

Reference herein to a fluorescent ligand is to a ligand whose pharmacological properties are known and which is associated with a fluorescent moiety but nevertheless maintains its pharmacological properties, e.g. binding affinity and functional activity, on binding to a cell surface receptor. Reference herein to a long acting ligand is to a ligand which remains bound for a sufficient time to enable identification and sorting as hereinbefore defined.

Reference herein to a reversible fluorescent ligand is to a ligand which binds to a cell surface receptor for a period and ultimately dissociates therefrom. Suitably binding is for a period sufficient to allow sorting by flow cytometry.

Reference herein to a specific or particular level of expression is to a specific uniform level of expression. Expression levels are manifested in terms of fluorescence brightness or fluorescence intensity. In the process of flow cytometry, filters can be applied to distinguish cells exhibiting low, medium or high level intensity respectively. Absolute values, of intensity cannot be given as they are dependent on each individual example, and are assessed in each case on a relative scale. Preferably therefore the method of the invention is identification of single cells expressing the receptor protein at a level corresponding to a low, medium or high fluorescence intensity, ie within the lowest 20-40%, more preferably the lowest 33% intensity, the medium 20-40%, more preferably 33% intensity or the highest 20-40%, more preferably the highest 33% intensity. Preferably the intensity is determined with respect to all single cells in the sample.

The present invention enables generation of a recombinant clonal cell line expressing a target cell surface receptor at a specific level, using (reversibly binding) fluorescent ligands which do not deactivate the cell surface receptor. The GFP label is a permanent part of the receptor when it is expressed. The advantage of the present invention is that the native or wild type unmodified receptor can be expressed and monitored using the fluorescent ligand. In the case of the present invention, whether or not the ligand is reversibly binding, ie whether it dissociates from the receptor or not, the expanded cell line is identical to the native or wild type receptor. In the case of GFP labelling the finished product is a cell line comprising GFP-tagged receptor.

Preferably a cell surface receptor is selected from G protein-coupled receptors (GPCRs), ligand-gated ion channels and tyrosine kinase receptors.

G protein coupled receptors are single chain proteins that cross the cell membrane seven times. The N terminus is outside of the cell and the C terminus is on the cytosolic side of the receptor protein. These receptors primarily mediate their effects by interaction with heterotrimeric G proteins.

Preferably a GPCR is selected from an adenosine receptor, a beta-adrenoceptor, a muscarinic receptor, a histamine receptor, an opiate receptor, a cannabinoid receptor, a chemokine receptor, an alpha-adrenoceptor, a GABA receptor, a prostanoid receptor, a 5-HT (serotonin) receptor, an excitatory aminoacid receptor (e.g. glutamate), a dopamine receptor, a protease-activating receptor, a neurokinin receptor, an angiotensin receptor, an oxytocin receptor, a leukotriene receptor, a nucleotide receptor (purines and pyrimidines), a calcium-sensing receptor, a thyroid-stimulating hormone receptor, a neurotensin receptor, a vasopressin receptor, an olfactory receptor, a nucleobase receptor (e.g. adenosine), a lysophosphatidic acid receptor, a sphingolipid receptor, a tyramine receptor (trace amines), a free-fatty acid receptor and a cyclic nucleotide receptor or the like. Most preferably a GPCR is selected from cannabinoid, metabotropic glutamate, dopamine and muscarinic acetylcholine receptors, most preferably CB₁, mGlu₅, D1 and M₃.

Ion channels may be classified by gating, i.e. what opens and closes the channels. Voltage-gated ion channels activate/inactivate depending on the voltage gradient across the plasma membrane, while ligand-gated ion channels activate/inactivate depending on binding of ligands to the channel. Ligand-gated ion channels are made up of several subunits that are organised in the plasma membrane to create a pore through which transport cations or anions can move. The ligand binding site is normally located at the interface between subunits.

Not all ion-channels are receptors. It will therefore be clear that the invention applies to those ion-channels which are receptors, i.e. recognise hormones. Preferably a ligand gated ion-channel is selected from cys-loop receptors GABA_{A}, GABA_{C}, Glycine (GlyR), Serotonine (5-HT), nicotinic acetylcholine (nAChR) receptors, ionotropic glutamate-gated receptors GluR, KA, NR1, NR2 and NR3, and ATP-gated P2X receptors and the like.

Tyrosine kinase receptors (e.g. those for various growth factors and insulin) normally work as a dimer and have an extracellular facing ligand binding site and an intracellular tyrosine kinase enzymic activity. Signalling is mediated as a result of phosphorylation of tyrosine residues and the induction of a cascade of protein phosphorylation events.

Tyrosine kinases are also divided into receptor (RTK) and non-receptor types. Preferably a tyrosine kinase receptor is selected from an EGF receptor, an insulin receptor, a PDGF receptor, an NGF receptor, an FGF receptor, a VEGF receptor, an HGF receptor, a Trk receptor and a TIE receptor.

Preferably a fluorescent ligand comprises any fluorophore coupled to a ligand specific to any of the above defined receptors. More preferably a fluorescent ligand is selected from those disclosed in WO 2004088312 and WO2006032926, the contents of which are incorporated herein by reference, and from novel ligand-linkers as hereinbelow defined in combination with a suitable fluorophore, more preferably with BODIPY 630-650 shown below.

In WO2004088312 we disclose fluorescent ligands (agonists and antagonists) for a number of G-protein coupled receptors. Using confocal microscopy we have been able to show that these bind selectively to membrane receptors in single living cells. Furthermore using fluorescence correlation spectroscopy (FCS) we have been able to evaluate quantitatively the characteristics of this binding in small microdomains of the membrane of single living cells.

More preferably a fluorescent ligand for use in the method of the invention is identified by the methodology of WO 2004088312 as summarised in WO 2006032926 for determining the functional response or pharmacological properties of a fluorescent ligand, comprising:
a) priming a cell or cell material with a sensor for a biological response;
b) subsequently contacting with a fluorescent ligand
   wherein the binding of the fluorescent ligand and its associated biological response are detected or monitored in the same cell and are distinct allowing separate readout, and wherein if binding, and therefore fluorescence, of the fluorescent ligand is detected, and if the associated measurable biological response from the cell or cell material is maintained, this indicates that the fluorescent ligand is a potential agonist, or if the associated measurable biological response from the cell or cell material is reduced or is absent, this indicates that the fluorescent ligand is a potential neutral antagonist or inverse agonist.

Preferably a fluorescent ligand comprises one or a plurality of ligand moieties linked to one or a plurality of fluorescent moieties via a linker at a linking site which maintains ligand activity.

Preferably a ligand moiety for a GPCR in a fluorescent ligand of the invention or for use in the invention is selected from any compound which is effective as a ligand for an adenosine receptor, a beta-adrenoceptor, a muscarinic receptor, a histamine receptor, an opiate receptor, a cannabinoid receptor, a chemokine receptor, an alpha-adrenoceptor, a GABA receptor, a prostanoid receptor, a 5-HT (serotonin) receptor, an excitatory aminoacid receptor (e.g. glutamate), a dopamine receptor, a protease-activating receptor, a neurokinin receptor, an angiotensin receptor, an oxytocin receptor, a leukotriene receptor, a nucleotide receptor (purines and pyrimidines), a calcium-sensing receptor, a thyroid-stimulating hormone receptor, a neurotensin receptor, a vasopressin receptor, an olfactory receptor, a nucleobase receptor (e.g. adenosine), a lysophosphatidic acid receptor, a sphingolipid receptor, a tyramine receptor (trace amines), a free-fatty acid receptor and a cyclic nucleotide receptor or the like, preferably for a GPCR receptor for example a) an adenosine receptor antagonist b) an adenosine receptor agonist c) a beta-adrenoceptor agonist and d) a beta-adrenoceptor antagonist. Preferably a ligand is a non-peptide ligand.

A fluorescent moiety may be any moiety recited in WO 2004088312. Preferably a fluorescent moiety is any red, green, near ir, blue or the like dyes or other class of dye. Suitably a fluorescent ligand is selected from dyes in particular including fluorescein, fluorescein derivatives including FITC, and fluorescein-like molecules such as Oregon Green™ and its derivatives, Texas Red™, 7-nitrobenz-2-oxa-1,3-diazole (NBD) and derivatives thereof, coumarin and derivatives, naphthalene including derivatives of dansyl chloride or its analogues or derivatives, Cascade Blue™ EvoBlue and fluorescent derivatives thereof, pyrenes and pyridyloxazole derivatives, the cyanine dyes, the dyomics (DY dyes and ATTO dyes) and fluorescent derivatives thereof, the Alexafluor dyes and derivatives, BDI dyes including the commercially available Bodipy™ dyes, erythosin, eosin, FITC, pyrenes, anthracenes, acridines, fluorescent phycobiliproteins and their conjugates and fluoresceinated microbeads, Rhodamine and fluorescent derivatives thereof including Rhodamine Green™ including the tetramethylrhodamines, X-rhodamines and Texas Red derivatives, and Rhodol Green™ coupled to amine groups using the isocyanate, succinimidyl ester or dichlorotriazinyl-reactive groups and other red, blue or green fluorescent dyes in particular red dyes as reviewed in Buschmann V et al, Bioconjugate Chemistry (2002), ASAP article.

Preferred BODIPY™ (4,4-difluoro-4-bora-3a,4a-diaz-s-indacene) fluorophores include those which span the visible spectrum and include those listed in U.S. Pat. No. 4,774,339; U.S. Pat. No. 5,187,288; U.S. Pat. No. 5,248,782; U.S. Pat. No. 5,274,113; U.S. Pat. No. 5,433,896; U.S. Pat. No. 5,451,663. A preferred member of this group is selected from any heteroaryl substituted BODIPY™ dyes as described in the above patents the contents of which are incorporated herein by reference.

More preferably a fluorescent ligand comprises fluorescein, Texas Red™, Cy5.5 or Cy5 or analogues thereof, BODIPY™ 630/650 and analogues thereof, DY-630, DY-640, DY-650 or DY-655 or analogues thereof, ATTO 655 or ATTO 680 or analogues thereof, EvoBlue 30 or analogues thereof, Alexa 647 or analogues thereof.

Suitably, a fluorescent moiety is derived from any of the above commercially available fluorophores, comprising or modified to comprise a reactive group facilitating linking to a ligand.

Preferably the fluorescent ligand of the invention is tailored by the site of linking of fluorescent and ligand moieties, the means of linking, ie nature and length of linker, and the stoichiometry thereof, ie 1:1, 2:1, 1:2 etc, whereby binding and function of the ligand are retained in the fluorescent ligand, and pharmacological properties are known whereby modulation of binding and function are known.

Preferably a fluorescent ligand is of the formula:

LigJ_{L}LJ_{Fl}Fl

including salts thereof, which may be present as a racemate or as one of its optically active isomers
wherein Lig comprises a ligand moiety, Fl comprises a fluorescent moiety and L comprises a linker as hereinbefore defined and as defined in WO2004088312 and WO 2006032926, and wherein J_{Fl} and J_{L} comprise linking site or linking functionality as defined in WO2004088312 and WO 2006032926 (where J_{T} is J_{Fl}), the contents of which are incorporated herein by reference.

The fluorescent ligand for use in the invention may be a novel fluorescent ligand of the formula:

Lig₃J_{L}LJ_{Fl}Fl

wherein J_{L}, L, J_{Fl} and Fl are as hereinbefore defined and

Lig₃ is —X(Z)Ar₁(Y—Ar₂)ₐ

where

X is selected from C, CH or N

Z is selected from H, ═O, C₁ alk(ox)yl or a single bond linking X and Ar₂

Y is selected from C₁₋₆ alkoxyl such as —OCH₂—, a single bond, C₁₋₆ alkyl, C₁₋₆ amine, C₁₋₆ carbonyl, —NHC(═O)— and a 5-7 membered N-containing saturated heterocycle containing 1, 2 or 3 N atoms

a is a whole number integer 1 or 2

Ar₁ is a 5 or 6 membered (hetero)aromatic, wherein a heteroatom is N, optionally substituted by C₁₋₈ hydrocarbyl, halo, OH and the like, more preferably is selected from the following structures:

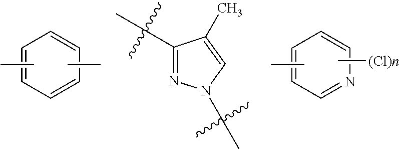

where n is selected from 0 and 1;
and Ar₂ is a 6 membered aromatic optionally substituted by C₁₋₈ hydrocarbyl, halo, OH and the like, more preferably is selected from the following structures:

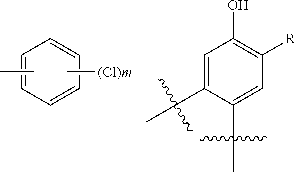

where m is selected from 0, 1 and 2 and R is C₁₋₈ hydrocarbyl, halo, OH and the like.

Preferably L is L₃ and is selected from C₁₋₁₂ alkyl, (C₁₋₆ alkoxy)₁₋₅₀C₁₋₁₂alkyl, amide and polyamide moieties including (C₁₋₁₂alkylNHCOC₁₋₁₂alkyl)₁₋₆₀, (C₁₋₁₂alkylCONHC₁₋₁₂alkyl)₁₋₅₀, (COCl₁₋₁₂alkylNH)₁₋₅₀, (NHC₁₋₁₂alkylCO)₁₋₅₀, 5-7 ring heterocyclyl C₁₋₁₂ alk(ox)yl, C₁₋₁₂ alk(ox)yl-5-7 ring heterocyclyl, and C₁₋₁₂ alk(ox)yl 5-7 ring heterocyclyl C₁₋₁₂ alkyl, wherein 1 to 3 heteroatoms, preferably 1 or 2 heteroatoms are selected from N, O and S.

More preferably L₃ is selected from the above where:

Alk(ox)yl is meth(ox)yl, eth(ox)yl, prop(ox)yl, but(ox)yl, pent(ox)yl, hex(ox)yl; hept(ox)yl; oct(ox)yl, non(ox)yl, dec(ox)yl, undec(ox)yl or dodec(ox)yl;

heterocyclyl rings comprise 6 ring atoms, more preferably are selected from piperazinyl, piperidinyl, 1,4-dioxane, 1,4-dithiane, morpholine and thiomorpholine;
(C₁₋₆ alkoxy)₁₋₅₀ are (ethoxy), (ethoxyethoxy), (methoxy), (methoxymethoxy) or a combination thereof;
amide and polyamide moieties are (COethyleneNH)₁₋₂, C₂₋₄ alkyl NHCO C₂₋₄alkyl, C₂₋₄ alkyl CONH C₂₋₄alkyl, most preferably selected from COethyleneNH, C₂ alkyl NHCO C₂ alkyl, C₂ alkyl NHCO C₄ alkyl, C4 alkyl NHCO C₂ alkyl, C₂ alkyl CONH C₂alkyl, C₂ alkyl CONH C₄alkyl and C₄ alkyl CONH C₂alkyl.

Preferably J_{L} and J_{Fl} are selected from NH, CO, 5-7 ring heterocyclyl wherein 2 to 3 heteroatoms, preferably 2 heteroatoms are selected from N and O. Preferably a 5-7 ring heterocyclyl is selected from piperazine and 1,4-dioxane. Preferably J_{L} and J_{Fl} are both NH, one is NH and the other is CO, or one is NH and the other is 5-7 ring heterocyclyl.

More preferably a novel fluorescent ligand for use in the invention is selected from the following compounds illustrated in part in FIG. 1 annexed hereto:

### M₃ Type Ligands

N-{2-[4-(2-oxo-2-(6-oxo-5,6-dihydro-11H-pyrido[2,3-b][1,4]benzodiazepine-11-yl)ethyl)-piperazine-1-yl]ethyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{4-[4-(2-oxo-2-(6-oxo-5,6-dihydro-11H-pyrido[2,3-b][1,4]benzodiazepine-11-yl)ethyl)-piperazine-1-yl]butyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{8-[4-(2-oxo-2-(6-oxo-5,6-dihydro-11H-pyrido[2,3-b][1,4]benzodiazepine-11-yl)ethyl)-piperazine-1-yl]octyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{2-[2-(2-(4-(2-oxo-2-(6-oxo-5,6-dihydro-11H-pyrido[2,3-b][1,4]benzodiazepine-11-yl)ethyl)-piperazine-1-yl)ethoxy)ethoxy]ethyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide

### D1-Type Ligands

N-{3-oxo-3-[4-(8-methoxy-3-methyl-2,3,4,5-tetrahydro-1H-3-benzazepine-1-yl)phenylamino]propyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{5-oxo-5-[4-(8-methoxy-3-methyl-2,3,4,5-tetrahydro-1H-3-benzazepine-1-yl)phenylamino]pentyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{6-oxo-6-[4-(8-methoxy-3-methyl-2,3,4,5-tetrahydro-1H-3-benzazepine-1-yl)phenylamino]hexyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{8-oxo-8-[4-(8-methoxy-3-methyl-2,3,4,5-tetrahydro-1H-3-benzazepine-1-yl)phenylamino]octyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{4-aza-3,7-dioxo-7-[4-(8-methoxy-3-methyl-2,3,4,5-tetrahydro-1H-3-benzazepine-1-yl)phenylamino]heptyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{4-aza-3,7-dioxo-7-[4-(7-bromo-8-methoxy-3-methyl-2,3,4,5-tetrahydro-1H-3-benzazepine-1-yl)phenylamino]heptyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
   mGluR5-Type Ligands
N-{3-aza-4-oxo-4-[2-chloro-6-(3-chlorobenzyloxy)pyridine-4-yl]butyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}-hexanamide
N-{5-aza-6-oxo-6-[2-chloro-6-(3-chlorobenzyloxy)pyridine-4-yl]hexyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}-hexanamide
N-{9-aza-10-oxo-10-[2-chloro-6-(3-chlorobenzyloxy)pyridine-4-yl]decyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}-hexanamide
N-{9-aza-3,6-dioxa-10-oxo-10-[2-chloro-6-(3-chlorobenzyloxy)pyridine-4-yl]decyl}-6-{2-[4-(2-(4,4-difluoro-4,4a-dihydro-5-(thiophen-2-yl)-4-bora-3a,4a-diaza-s-indacene-3-yl)vinyl)phenoxy]-acetamido}hexanamide
N-{4,7-diaza-3,8-dioxo-8-[2-chloro-6-(3-chlorobenzyloxy)pyridine-4-yl]octyl}-6-{2-[4-(2-(4,4-difluoro-4,4a-dihydro-5-(thiophen-2-yl)-4-bora-3a,4a-diaza-s-indacene-3-yl)vinyl)phenoxy]-acetamido}hexanamide
N-{4,9-diaza-3,10-dioxo-10-[2-chloro-6-(3-chlorobenzyloxy)pyridine-4-yl]decyl}-6-{2-[4-(2-(4,4-difluoro-4,4a-dihydro-5-(thiophen-2-yl)-4-bora-3a,4a-diaza-s-indacene-3-yl)vinyl)phenoxy]-acetamido}hexanamide
N-{6,9-diaza-5,10-dioxo-10-[2-chloro-6-(3-chlorobenzyloxy)pyridine-4-yl]decyl}-6-{2-[4-(2-(4,4-difluoro-4,4a-dihydro-5-(thiophen-2-yl)-4-bora-3a,4a-diaza-s-indacene-3-yl)vinyl)phenoxy]-acetamido}hexanamide

### CB1-Type Ligands

N-{3-aza-4-oxo-4-[5-(4-chlorophenyl)-1-(2,4-dichlorophenyl)-4-methyl-1H-pyrazole-3-yl]butyl}-6-{2-[4-(2-(4,4-difluoro-4,4a-dihydro-5-(thiophen-2-yl)-4-bora-3a,4a-diaza-s-indacene-3-yl)vinyl)-phenoxy]acetamido}hexanamide
N-{5-aza-6-oxo-6-[5-(4-chlorophenyl)-1-(2,4-dichlorophenyl)-4-methyl-1H-pyrazole-3-yl]hexyl}-6-{2-[4-(2-(4,4-difluoro-4,4a-dihydro-5-(thiophen-2-yl)-4-bora-3a,4a-diaza-s-indacene-3-yl)vinyl)-phenoxy]acetamido}hexanamide
N-{9-aza-10-oxo-10-[5-(4-chlorophenyl)-1-(2,4-dichlorophenyl)-4-methyl-1H-pyrazole-3-yl]-decyl}-6-{2-[4-(2-(4,4-difluoro-4,4a-dihydro-5-(thiophen-2-yl)-4-bora-3a,4a-diaza-s-indacene-3-yl)vinyl)-phenoxy]acetamido}hexanamide
N-{2-[4-(1-oxo-1-(5-(4-chlorophenyl)-1-(2,4-dichlorophenyl)-4-methyl-1H-pyrazole-3-yl)methyl)-piperazine-1-yl]ethyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{4-[4-(1-oxo-1-(5-(4-chlorophenyl)-1-(2,4-dichlorophenyl)-4-methyl-1H-pyrazole-3-yl)methyl)-piperazine-1-yl]butyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{8-[4-(1-oxo-1-(5-(4-chlorophenyl)-1-(2,4-dichlorophenyl)-4-methyl-1H-pyrazole-3-yl)methyl)-piperazine-1-yl]octyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{9-aza-3,6-dioxa-10-oxo-10-[5-(4-chlorophenyl)-1-(2,4-dichlorophenyl)-4-methyl-1H-pyrazole-3-yl]decyl}-6-{2-[4-(2-(4,4-difluoro-4,4a-dihydro-5-(thiophen-2-yl)-4-bora-3a,4a-diaza-s-indacene-3-yl)vinyl)-phenoxy]acetamido}hexanamide
N-{3,6-dioxa-8-[4-(1-oxo-1-(5-(4-chlorophenyl)-1-(2,4-dichlorophenyl)-4-methyl-1H-pyrazole-3-yl)methyl)piperazine-1-yl]octyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{4,9-diaza-3,10-dioxo-10-[5-(4-chlorophenyl)-1-(2,4-dichlorophenyl)-4-methyl-1H-pyrazole-3-yl]decyl}-6-{2-[4-(2-(4,4-difluoro-4,4a-dihydro-5-(thiophen-2-yl)-4-bora-3a,4a-diaza-s-indacene-3-yl)vinyl)-phenoxy]acetamido}hexanamide

A fluorescent ligand may have affinity such that it binds permanently, semi-permanently or transiently, and may remain bound or dissociate prior to or during expansion of cell lines. In a particular advantage of the invention a fluorescent ligand binds semi-permanently or transiently, for a sufficient period to allow binding and sorting by flow cytometry as hereinbefore defined. Such period may suitably be of the order of seconds, more preferably minutes, up to 1 hour. Suitably binding is for a period of 30 minutes to an hour.

Flow cytometry, as hereinbefore defined, is the rapid sequential analysis of single cells, usually using laser light and fluorescent labels, and identification of single cells with a particular fluorescence intensity (at a particular fluorescence wavelength) which can then be sorted into populations with a particular level of fluorescence intensity or into single cells in a multiwell plate. For example, the Beckman-Coulter Epics Altra is a cytometry sorter, which is equipped with 3 lasers, allowing excitation with ultraviolet or violet, blue and red light. Laser beams can be aligned to strike a cell simultaneously, or they can be offset so that fluorescence from each laser can be separated in time. There are eight detectors, allowing analysis of 6 colours and scatter parameters. Cells identified using this powerful analysis capability can if required be sorted, using an electrostatic deflection mechanism. Analysis/sort rates of over 5000 cells per second are possible, and sorted cells can then be placed into wells of microplates. The instrument is fitted with a robotic arm allowing the programmed deposition of specified numbers of particular cell types, for example into each well of a 96 well plate. Sorting purity should be 99%.

In a particular advantage of the present invention, fluorescent ligands can be used to both monitor the homogeneity of a cell population expressing a particular cell surface receptor (see Example 1 below) and also to provide the fluorescent signal for cytometry-based cell sorting.

Preferably the method of the invention includes identifying a ligand which is suitable for drug targets selected from GPCRs, ligand-gated ion channels and tyrosine kinase receptors. The method also includes subsequently screening compounds or compound libraries against the cell line of the invention in the presence and absence of the identified ligand.

A cell or cell material may comprise one or more cells, cell extracts, cell homogenates, purified or reconstituted proteins, recombinant proteins or synthesised proteins and the like, and includes a target receptor. Samples comprising cell material may be derived from plants, animals, fungi, protists, bacteria, archae or cell lines derived from such organisms. Animal or plant cells used to prepare the sample may be healthy or disfunctional and are optionally used in the diagnosis of a disease such as leukaemia or cancer. In a preferred embodiment of the invention the sample comprises mammalian cells, extracts and homogenates thereof.

Preferably a sample comprises live cell material, more preferably including individual cells or sub cell compartments, most preferably comprising GPCRs, ligand-gated ion channels and tyrosine kinase receptors in living cells, membrane containing these proteins, solubilised receptors, or channels or GPCR arrays. Cell material may be obtained in known manner by culturing cells or by expressing proteins in cells.

In a preferred embodiment the cell material is a cell expressing a GPCR, ligand-gated ion channel or tyrosine kinase receptor as hereinbefore defined, more preferably CHO-cells expressing the same.

Cell material may be tagged prior to contact with the fluorescent ligand, for example by tagging with GFP, for example GFP tagged GPCR's, GFP tagged (ligand-gated) ion channels and GFP tagged tyrosine kinase receptors, or a native receptor or ligand-gated ion channel to which a fluorescent antibody has been targetted, to allow visualising of the cell receptors or ion channels, and overlay with the fluorescent ligands.

Receptors may be provided in membrane samples or in acutely dispersed cell samples, for example endogenous receptors such as A₁-AR in acutely dispersed cells. The adenosine receptor binding site is located deep within the pocket of the receptor, whereby a fluorescent ligand with linker is a preferred fluorescent (ant)agonist. Whilst there is considerable freedom in modifying the ligand and retaining antagonist binding activity, it is harder to retain agonist activating activity, ie. activating the receptors functions on binding.

In a particular advantage of the invention the fluorescent ligands are suitable for use in combination with FCS enabling the study of ligand-receptor binding at the single molecule level. Because of the nature of the events being monitored FCS is ideal for the study of thermodynamic and kinetic features of molecular interactions in solution. Another particular advantage of the invention is that the FCS approach can be adapted to monitor ligand-receptor binding at the single molecule level using photon counting fluorescence intensity measurements. This removes any requirement for the molecules to be moving within the confocal volume.

With ligands showing low background fluorescence it is not necessary to remove unbound ligand by washing before performing either confocal microscopy or FCS. It is therefore possible to measure fluorescence with time, in both time and concentration dependent manner.

Confocal microscopy (CSLM) allows visualisation of a section through a cell showing concentration of fluorophore at the cell edges indicating membrane receptor binding. Visualisation is of a particular plane of focus such that a “slice” through an individual cell may be observed, as known in the art. Different coloured channels may be selected to visualise different fluorophore types.

FCS is a non-invasive technique which analyses the diffusion characteristics of fluorescent species through a very small excitation volume (<10⁻¹⁵ I) by statistically analysing the pattern of their photon emissions. Thus fast-diffusing free ligand can be distinguished from slowly-diffusing receptor-bound ligand and quantified simultaneously when the volume is localised to the cell membrane. Preferably the method incorporating FCS comprises measuring fluctuations in fluorescence intensity in a confocal volume of <10⁻¹⁵ I. Statistical analysis of these fluctuations gives information about the speed of diffusion (i.e. mass) and concentration of the fluorescent molecules present. Thus free ligand (fast diffusing) and bound ligand (slow diffusing) can be quantified simultaneously on a single cell.

FCS (fluorescence correlation spectroscopy) correlates fluctuations in fluorescence emission of particles to parameters such as particle mass and concentration for the study of molecular interactions in solution. FCS essentially monitors spontaneous fluorescence intensity fluctuations of fluorescently tagged molecules in a microscopic detection volume (10⁻¹⁵ I) through analysis by a tightly focused laser beam.

In a further aspect of the invention there is provided a novel fluorescent ligand as hereinbefore defined. Preferably a novel fluorescent ligand is of formula

Lig₃J_{L}LJ_{T}Fl

as hereinbefore defined.

More preferably a novel fluorescent ligand is selected from compounds listed in FIG. 1 annexed hereto and as hereinbefore recited.

Further aspects of the invention are as hereinbefore defined.

In the method of the invention or the kit for use therewith as hereinbefore defined, a fluorescent ligand is preferably associated with information on its receptor binding, in order to select a suitable fluorescent ligand for a clonal cell line which it is desired to establish.

In a preferred use of a recombinant cell line obtained by the method of the invention, compound libraries may be screened and results directly compared, or receptor binding may be studied and pharmacology and signalling characteristics directly compared, by virtue of the uniform expression level of cells in the cell line.

In a further aspect of the invention there is provided the use of a known or novel fluorescent ligand, as hereinbefore defined or as defined in WO2004088312 or WO2006032926 or other publications, the contents of which are incorporated herein by reference, in the method of the invention.

In a further aspect of the invention there is provided a ligand identified by the method of the invention for drug targets selected from GPCRs, ligand-gated ion channels and tyrosine kinase receptors.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The invention is now illustrated in non limiting manner with reference to the Figures and examples.

### Example 1

### Synthesis of Novel Fluorescent Ligands

The hereinbefore recited compounds were synthesised using the method shown in the attached Schemes (FIG. 2a-d) and analysed (data given below):

### Example 1a

### Preparation of M3 Fluorescent Ligands—See Scheme FIG. 2a

### 11-(Chloroacetyl)-5,11-dihydro-6H-pyrido[2,3-b][1,4]benzodiazepine-6-one (1)

### General Procedure for Synthesis of (3)

To 11-(chloroacetyl)-5,11-dihydro-6H-pyrido[2,3-b][1,4]benzodiazepine-6-one (1) (1 equiv) was added a solution of amine linker (2) (1.3 equiv)- and N,N-diisopropylethylamine (3 equiv) in anhydrous acetonitrile and the reaction heated under an atmosphere of nitrogen at 85° C. for 16-18 h. The reaction mixtures were filtered, the filtrate evaporated under reduced pressure and the crude compounds purified by flash column chromatography or preparative thin layer chromatography on silica using 5 to 15% methanol in dichloromethane as eluent to give the protected amines (3).
11-{[4-(2-Benzyloxycarbonylaminoethyl)piperazine-1-yl]acetyl}-5,11-dihydro-6H-pyrido[2,3-b]-[1,4]benzodiazepine-6-one (3a)
11-{[4-(4-Benzyloxycarbonylaminobutyl)piperazine-1-yl]acetyl}-5,11-dihydro-6H-pyrido[2,3-b]-[1,4]benzodiazepine-6-one (3b)

m/z (TOF ES+) found 543 (MH⁺, 100%).
11-{[4-(8-Azidooctyl)piperazine-1-yl]acetyl}-5,11-dihydro-6H-pyrido[2,3-b]-[1,4]benzodiazepine-6-one (3c)
11-[(4-{2-[2(2-Azidoethoxy)ethoxy]ethyl}piperazine-1-yl)acetyl]-5,11-dihydro-6H-pyrido-[2,3-b][1,4]benzodiazepine-6-one (3d)

### General Procedure for Synthesis of (4)

To a solution of the benzylcarbamate or azide (3) (1 equiv) in methanol was added ammonium formate (5 equiv) followed by 10% palladium on carbon (100 w/w % with benzylcarbamate or azide). The reaction mixture was stirred at room temperature for 3 h, filtered through celite and the filter washed with dichloromethane (×3). The filtrate was evaporated under reduced pressure and to the resultant residue was added saturated aqueous sodium bicarbonate which was then extracted with dichloromethane (×4). The combined organic extracts were dried over anhydrous magnesium sulphate and evaporated under reduced pressure to give the amines (4) which were used without further purification.
11-{[4-(2-Aminoethyl)piperazine-1-yl]acetyl}-5,11-dihydro-6H-pyrido[2,3-[4-(1,4]benzodiazepine-6-one (4a)
11-{(4-(4-Aminobutyl)piperazine-1-yl]acetyl}-5,11-dihydro-6H-pyrido[2,3-b]-[1,4]benzodiazepine-6-one (4b)
11-{[4-(8-Aminooctyl)piperazine-1-yl]acetyl}-5,11-dihydro-6H-pyrido[2,3-b]-[1,4]benzodiazepine-6-one (4c)
11-[(4-{2-[2(2-Aminoethoxy)ethoxy]ethyl}piperazine-1-yl)acetyl]-5,11-dihydro-6H-pyrido-[2,3-b][1,4]benzodiazepine-6-one (4d)

### General Procedure for Synthesis of (5)

BODIPY 630/650-X, SE (1 equiv) and amine (4) (3-4 equiv) were dissolved in dichloromethane and stirred in the dark for 2-5 h. The solvent was evaporated under reduced pressure and the crude mixture was purified by preparative thin layer chromatography on silica using 15:85 methanol/dichloromethane as eluent to give compounds (5) as blue solids.
N-{2-[4-(2-oxo-2-(6-oxo-5,6-dihydro-11H-pyrido[2,3-b][1,4]benzodiazepine-11-yl)ethyl)-piperazine-1-yl]ethyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide (5a)

m/z (TOF ES+) found 926 (MH⁺, 90%), 129 (100).
N-{4-[4-(2-oxo-2-(6-oxo-5,6-dihydro-11H-pyrido[2,3-b][1,4]benzodiazepine-11-yl)ethyl)-piperazine-1-yl]butyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide (5b)

m/z (TOF ES+) found 954 (MH⁺, 100%).
N-{8-[4-(2-oxo-2-(6-oxo-5,6-dihydro-11H-pyrido[2,3-b][1,4]benzodiazepine-11-yl)ethyl)-piperazine-1-yl]octyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide (5c)

m/z (TOF ES+) found 1010 (MH⁺, 100%).
N-{2-[2-(2-(4-(2-oxo-2-(6-oxo-5,6-dihydro-11H-pyrido[2,3-b][1,4]benzodiazepine-11-yl)ethyl)-piperazine-1-yl)ethoxy)ethoxy]ethyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide (5d)

m/z (TOF ES+) found 1014 (MH⁺, 100%).

### Example 1b

### Preparation of D1 Fluorescent Ligands—See Scheme FIG. 2b

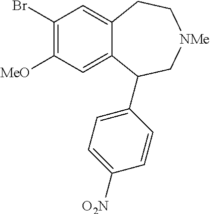

8-Methoxy-7-bromo-3-methyl-1-(4′-nitrophenyl)-2,3,4,5-tetrahydro-1H-3-benzazepine. To a solution of 8-methoxy-3-methyl-1-(4′-nitrophenyl)-2,3,4,5-tetrahydro-1H-3-benzazepine (Neumeyer, J. L.; Baindur, N.; Yuan, J.; Booth, G.; Seeman, P.; Nizniki, H. B. J. Med. Chem. 1990, 33, 521) (109 mg, 349 μmol) in CHCl₃ (2 mL) and THF (2 mL) was added bromine (54 μL, 1.05 mmol). The reaction was stirred at room temperature for 2 h and then quenched by the addition of a 10% aqueous solution of sodium metabisulfite (10 mL) and the aqueous was then extracted with CH₂Cl₂ (3×20 mL). The combined organic extracts were dried over anhydrous MgSO₄ and evaporated under reduced pressure. This crude mixture was partially purified by automated column chromatography on silica using a gradient of MeOH/CH₂Cl₂ (0:100 MeOH/CH₂Cl₂ to 5:95 MeOH/CH₂Cl₂) as eluent, followed by a second purification by PTLC on silica using 3:97 MeOH/CH₂Cl₂ as eluent to give the title compound (61 mg, 45%) as an off white solid. ¹H NMR (400 MHz, CDCl₃) 2.34 (3H, s, NCH₃), 2.43-2.52 (1H, m, CHH), 2.60-2.73 (2H, m, CH₂), 2.80-2.96 (2H, m, CH₂), 3.03-3.13 (1H, m, CHH), 3.62 (3H, s, OCH₃), 3.33 (1H, br d, J=6.5, CHCH₂), 6.21 (1H, s, CH), 7.29 (1H, s, CH), 7.30 (2H, d, J=9.1, CH), 8.16 (2H, d, J=9.1, CH).

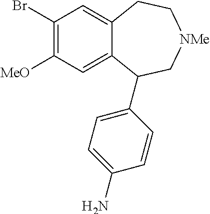

8-Methoxy-7-bromo-3-methyl-1-(4′-aminophenyl)-2,3,4,5-tetrahydro-1H-3-benzazepine. To a solution of 8-methoxy-7-bromo-3-methyl-1-(4′-nitrophenyl)-2,3,4,5-tetrahydro-1H-3-benzazepine (37 mg, 94.6 μmol) in EtOH (2 mL) and concentrated ammonia (2 mL) was added Na₂S₂O₄ (165 mg, 946 μmol) and this mixture was stirred at room temperature for 3 h. The reaction was concentrated under reduced pressure and CH₂Cl₂ (5 mL) was added to the aqueous which was taken to pH 8 with concentrated HCl. The layers were separated, the aqueous was extracted with CH₂Cl₂ (3×5 mL) and the combined organic extracts were dried over anhydrous MgSO₄ and evaporated under reduced pressure to give the title compound (15 mg, 44%) as an off white solid. MS (TOF ES+) 363 (MH⁺, 100%), 361 (93).

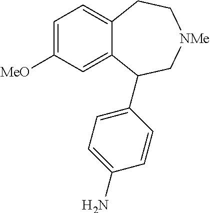

8-Methoxy-3-methyl-1-(4′-aminophenyl)-2,3,4,5-tetrahydro-1H-3-benzazepine. To a solution of 8-methoxy-3-methyl-1-(4′-nitro phenyl)-2,3,4,5-tetrahydro-1H-3-benzazepine^{Error! Bookmark not defined.} (501 mg, 968 μmol) in MeOH (10.8 mL) was added H₂O (1.2 mL) followed by 10% Pd/C (250 mg). The reaction flask was briefly evacuated and hydrogen was added via a balloon; this mixture was then stirred at room temperature for 17 h. The reaction mixture was filtered through celite, and the filter was washed with CH₂Cl₂ (2×15 mL). The solvent was evaporated under reduced pressure to give the title compound (395 mg, 87%) as a dark orange solid. ¹H NMR (400 MHz, CDCl₃) 2.34-2.48 (1H, m, CHH), 2.43 (3H, s, NCH₃), 2.76-2.90 (2H, m, CH₂), 2.91-3.05 (1H, m, CHH), 3.09-3.27 (2H, m, CH₂), 3.65 (3H, s, OCH₃), 4.30 (1H, br d, J=8.4, CHCH₂), 6.29 (1H, d, J=2.5, CH), 6.63 (1H, dd, J=8.2, 2.6, CH), 6.66-6.73 (2H, m), 6.94-6.99 (2H, m), 7.04 (1H, d, J=8.2, CH); MS (TOF ES+) 283 (MH⁺, 100%).

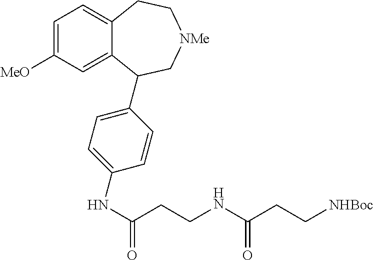

8-Methoxy-3-methyl-1-{[(4′-aminophenyl)-3-oxopropylamino]-3-oxopropyl-tert-butylcarbamate}-2,3,4,5-tetrahydro-1H-3-benzazepine. To a solution of Boc-β-Ala-β-Ala-OH (454 mg, 1.75 mmol) in CH₂Cl₂ (3 mL) was added DCC (360 mg, 1.75 mmol), DMAP (213 mg, 1.75 mmol) and Et₃N (243 μL, 1.75 mmol) sequentially. This mixture was stirred for 15 mins prior to the addition of a solution of 8-methoxy-3-methyl-1-(4′-aminophenyl)-2,3,4,5-tetrahydro-1H-3-benzazepine (248 mg, 880 μmol) in CH₂Cl₂ (3 mL), this mixture was stirred at room temperature for 14 h. The mixture was filtered through celite, washed with CH₂Cl₂ (2×10 mL) and the filtrate was evaporated under reduced pressure. This crude mixture was partially purified by automated column chromatography on silica using a gradient of MeOH/CH₂Cl₂ (5:95 MeOH/CH₂Cl₂ to 25:75 MeOH/CH₂Cl₂) as eluent, the resultant red oil was then purified by column chromatography on silica using (15:85 MeOH/CH₂Cl₂) as eluent to give the title compound (209 mg, 45%) as a red solid. ¹H NMR (400 MHz, CDCl₃) 1.43 (9H, s, C(CH₃)₃), 2.33-2.43 (3H, m), 2.39 (3H, s, NCH₃), 2.60 (2H, t, J=5.7, CH₂), 2.79 (1H, dd, J=14.4, 7.7, CHH), 2.87 (2H, br t, J=8.7, CH₂), 3.06 (2H, br d, J=12.7, CH₂), 3.40 (2H, dd, J=12.1, 6.0, CH₂NH), 3.62 (2H, dd, J=12.1, 6.0, CH₂NH), 3.66 (3H, s, OCH₃), 4.30 (1H, br d, J=8.0, CHCH₂), 5.15 (1H, br s, NH), 6.25 (1H, d, J=2.4, CH), 6.42-6.52 (1H, br m, NH), 6.64 (1H, dd, J=8.4, 2.4, CH), 7.05 (1H, d, J=8.4, CH), 7.14 (2H, d, J=8.0, 2×CH), 7.53 (2H, d, J=8.0, 2×CH), 8.06 (1H, br s, NH); HRMS (TOF ES+) Calc. for C₂₉H₄₁N₄O₅: 525.3077. Found: 525.3030 (MH⁺).

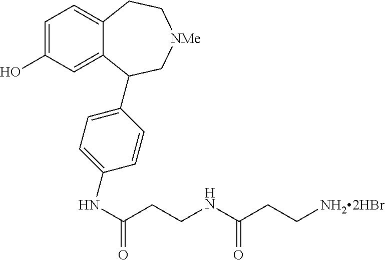

8-Hydroxy-3-methyl-1-{[(4′-aminophenyl)-3-oxopropylamino]-3-oxopropylamino}-2,3,4,5-tetrahydro-1H-3-benzazepine dihydrobromide. To a cooled (−78° C.) solution of 8-methoxy-3-methyl-1-{[(4′-aminophenyl)-3-oxopropylamino]-3-oxopropyl-tert-butylcarbamate}-2,3,4,5-tetrahydro-1H-3-benzazepine (32 mg, 61.1 μmol) in CH₂Cl₂ (1.2 mL) was added a solution of boron tribromide (611 μL, 611 μmol; 1 M in CH₂Cl₂) and the reaction mixture was allowed to return to room temperature and stirred under N₂ for 3 h. This mixture was then cooled to −78° C., quenched with MeOH (1 mL) and the solvent was evaporated under reduced pressure to give the title compound (29 mg, quant.) as a pale brown solid. MS (TOF ES+) 411 (MH⁺, 20%), 340 ([MH-βAla]⁺, 50%), 269 ([MH-2βAla]⁺, 100%).

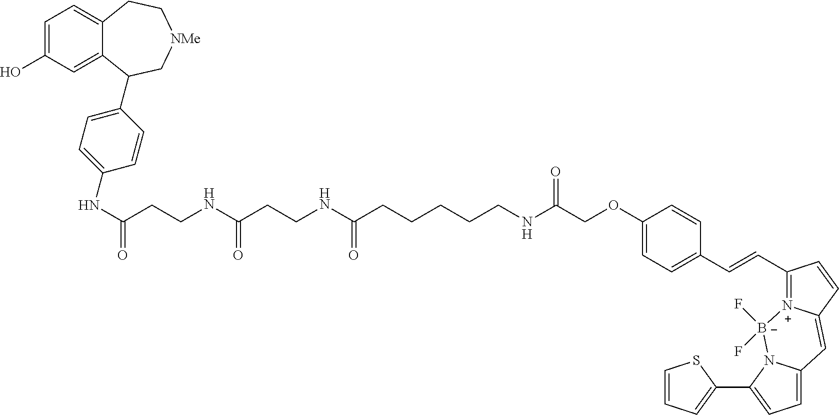

MAB-3-oxoaminopropyl-3-oxoaminopropyl-X-BY630. To BODIPY 630/650-X, SE (0.95 mg, 1.44 μmol) and 8-hydroxy-3-methyl-1-{[(4′-aminophenyl)-3-oxopropylamino]-3-oxopropylamino}-2,3,4,5-tetrahydro-1H-3-benzazepine dihydrobromide (5.3 mg, 9.27 μmol) was added a solution of DIPEA (3.5 μL, 20.2 μmol) in anhydrous DMF (1 mL) and this solution was stirred in the dark for 2 h. The solvent was evaporated under reduced pressure and this crude mixture was purified by PTLC on silica using (15:85 MeOH/CH₂Cl₂) as eluent to give the title compound (0.33 mg 21%) as a blue solid. MS (TOF ES+) 956 (MH⁺, 100%).

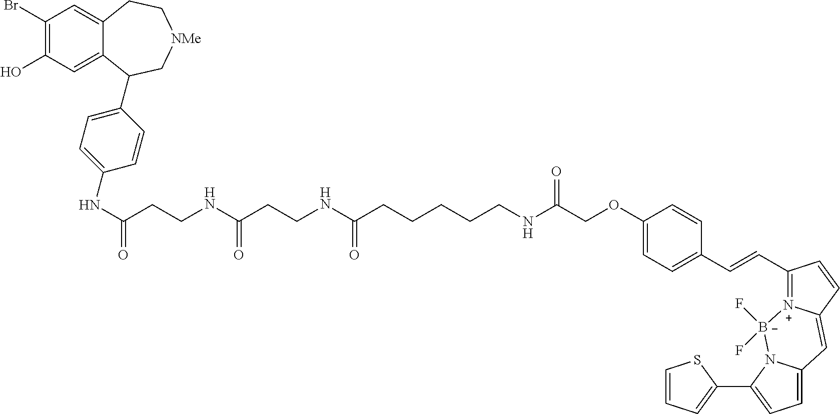

Br-MAB-3-oxoaminopropyl-3-oxoaminopropyl-X-BY630. HRMS (TOF ES+): Calc. for C₅₂H₅₆BBrF₂N₇O₆S: 1034.3257. Found: 1034.3292 (MH⁺).

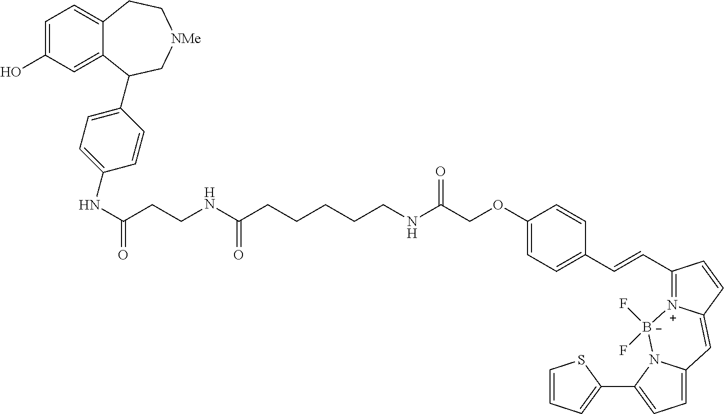

MAB-3-oxoaminopropyl-X-BY630. MS (TOF ES+) 885 (MH⁺, 100%).

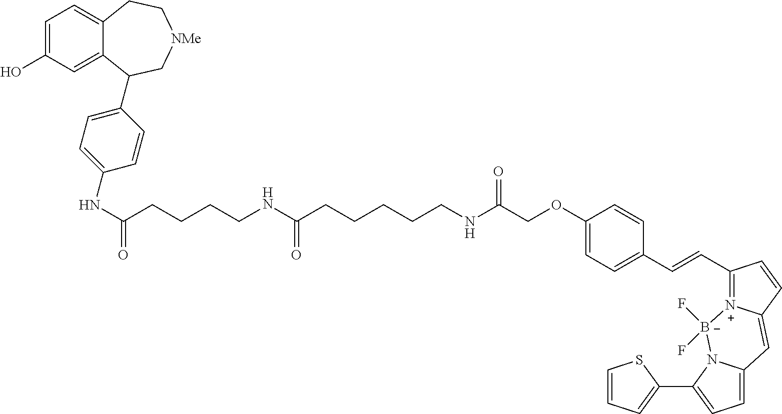

MAB-5-oxoaminopentyl-X-BY630. MS (TOF ES+) 913 (MH⁺, 100%).

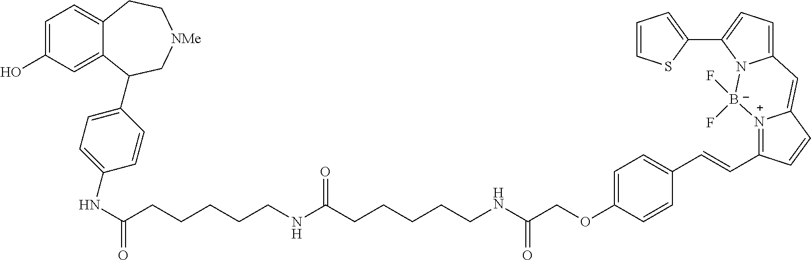

MAB-6-oxoaminohexyl-X-BY630. MS (TOF ES+) 927 (MH⁺, 100%).

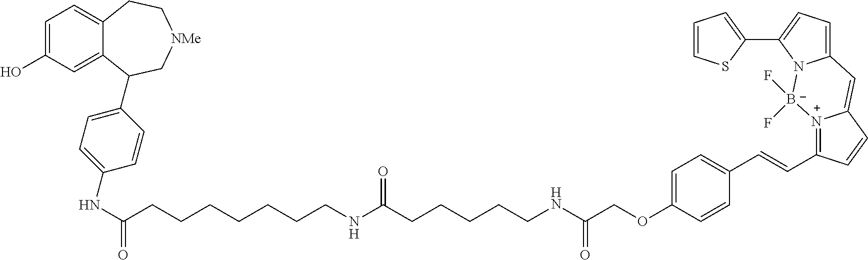

MAB-8-oxoaminooctyl-X-BY630. MS (TOF ES+) 955 (MH⁺, 100%).

### Example 1c

### Preparation of mGluR5 Fluorescent Ligands—See Scheme FIG. 2c

### Example 1d

### Preparation of CB1 Fluorescent Ligands—See Scheme FIG. 2d

### Example 2

A mixed population cell line expressing the human dopamine D1 receptor was screened with novel fluorescent dopamine D1 receptor antagonist N-{4-aza-3,7-dioxo-7-[4-(8-methoxy-3-methyl-2,3,4,5-tetrahydro-1H-3-benzazepine-1-yl)phenylamino]heptyl}-6-{2-[4-(2-(4,4-difluoro-4,4a-dihydro-5-(thiophen-2-yl)-4-bora-3a,4a-diaza-s-indacene-3-yl)vinyl), prepared as in Example 1. As can be clearly seen in FIG. 3, only about 20% of the cells label with the fluorescent D1-receptor antagonist indicating that only 20% of the cells are expressing the D1-receptor at their cell surface. Using FACS cell sorting, these cells are sorted and separated into populations of cells expressing low, medium or high levels of the dopamine D1 receptor. FIG. 4 shows the cell population obtained after sorting for medium expressing cells. A cell sorter such as the Beckman-Coulter Altra can then be used to put individual cells into single wells of a 96 well plate.

We claim:

1. A method for generating a clonal cell line expressing a target cell surface receptor, the method comprising:
(c) incubating a cell population expressing a known target cell surface receptor selected from G protein-coupled receptors (GPCRs), ligand-gated ion channels and tyrosine kinase receptors with a target cell surface receptor specific fluorescent non-peptide ligand or salt thereof, comprising a non-peptide ligand moiety for the target cell surface receptor that binds to the target cell surface receptor, linked to a fluorescent moiety;
(d) selecting single cells from step (c) expressing the target cell surface receptor at a given level of expression by monitoring the specific binding of the fluorescent ligand using flow cytometry; and additionally (e) expansion of selected single cells from step (d) into a clonal cell line.

2.-4. (canceled)

5. A method as claimed in claim 1 which comprises generating the cell population by means of
(a) transfecting cells with a plasmid encoding the cDNA sequence of the target receptor and an antibiotic selection marker; and
(b) antibiotic resistant selection of those cells expressing the particular cell surface receptor thereby generating a cell population.

6. A method as claimed in claim 1 wherein flow cytometry comprises the use of a fluorescence activated cell sorter (FACS™) or fluorescence activated cell sorting technique.

7.-8. (canceled)

9. A method as claimed in claim 1 wherein a fluorescent ligand is of the formula:
LigJ_{E}LJ_{Fl}Fl
or salts thereof, which may be present as a racemate or as one of its optically active isomers
wherein Lig comprises a ion-peptide ligand moiety specific to the known target cell surface receptor selected from G protein-coupled receptors (GPCRs), ligand-gated ion channels or tyrosine kinase receptors, Fl comprises a fluorescent moiety and L comprises a linker, and wherein J_{Fl} and J_{L} comprise linking site or linking functionality.

10. A method as claimed in claim 1 which includes identifying a ligand which is suitable for drug targets selected from GPCRs, ligand-gated ion channels and tyrosine kinase receptors by methodology for determining the functional response or pharmacological properties of a fluorescent ligand, comprising:
(a) priming a cell or cell material with a sensor for a biological response;
(b) subsequently contacting with a fluorescent ligand wherein the binding of the fluorescent ligand and its associated biological response are detected or monitored in the same cell and are distinct allowing separate readout, and wherein if binding, and therefore fluorescence, of the fluorescent ligand is detected, and if the associated measurable biological response from the cell or cell material is maintained, this indicates that the fluorescent ligand is a potential agonist, or if the associated measurable biological response from the cell or cell material is reduced or is absent, this indicates that the fluorescent ligand is a potential neutral antagonist or inverse agonist;
and optionally additionally subsequently screening compounds or compound libraries against the clonal cell line of claim 1 in the presence and absence of the identified ligand.

11. A long-acting reversibly-binding fluorescent ligand of the formula Lig₃J_{L}L₃J_{Fl}Fl
or salt thereof suitable for binding to a cell surface GPCR selected from cannabinoid, metabotropic glutamate, dopamine and muscarinic acetylcholine receptors in a cell population expressing such receptors, and for which it is desired to ascertain the level of expression, in the method of claim 1, whereby the specific binding of the fluorescent ligand is an indicator of the level of expression by individual cells in the cell population of the cell surface receptor, preferably as a reversible fluorescent tag for analysis and sorting, and identifying cells expressing a particular level of GPCR at the single cell level wherein Fl comprises a fluorescent moiety selected from a heteroaryl substituted 4,4-difluoro-4-bora-3a,4a-diaz-s-indacene fluorophore J_{Fl} and J_{L} comprise linking site or linking functionality and
Lig₃ is a non-peptide GPCR ligand moiety selected from a moiety of a cannabinoid, metabotropic glutamate, dopamine or muscarinic acetylcholine ligand, of formula —X(Z)Ar₁(Y—Ar₂)ₐ
where
X is selected from C, CH or N
Z is selected from H, ═O, C₁₋₆ alk(ox)yl or a single bond linking X and Ar₂
Y is selected from C₁₋₆ alkoxyl such as —OCH₂—, a single bond, C₁₋₆ alkyl, C₁₋₆ amine, C₁₋₆ carbonyl, —NHC(═O)— and a 5-7 membered N-containing saturated heterocycle containing 1, 2 or 3 N atoms
a is a whole number integer 1 or 2
Ar₁ is a 5 or 6 membered (hetero)aromatic, wherein a heteroatom is N, optionally substituted by C₁₋₈ hydrocarbyl, halo, OH and the like, more preferably is selected from the following structures: 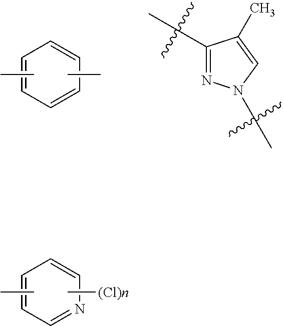
where n is selected from 0 and 1;
and Ar₂ is a 6 membered aromatic optionally substituted by C₁₋₈ hydrocarbyl, halo, OH and the like, more preferably is selected from the following structures: 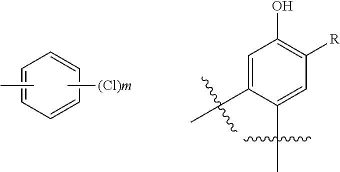
where m is selected from 0, 1 and 2 and R is C₁₋₈ hydrocarbyl, halo, OH and the like;

12. A ligand as claimed in claim 11 wherein L₃ is selected from C₁₋₁₂ alkyl, (C₁₋₆ alkoxy)₁₋₅₀C₁₋₁₂alkyl, amide and polyamide moieties including (C₁₋₁₂alkylNHCOC₁₋₁₂alkyl)₁₋₅₀, (C₁₋₁₂alkylCONHC₁₋₁₂alkyl)₁₋₅₀, (COC₁₋₁₂alkylNH)₁₋₅₀, (NHC₁₋₁₂alkylCO)₁₋₅₀, 5-7 ring heterocyclyl C₁₋₁₂ alk(ox)yl, C₁₋₁₂ alk(ox)yl-5-7 ring heterocyclyl, and C₁₋₁₂ alk(ox)yl 5-7 ring heterocyclyl C₁₋₁₂ alkyl, wherein 1 to 3 heteroatoms, preferably 1 or 2 heteroatoms are selected from N, O and S.

13.-14. (canceled)

15. A long-acting, reversibly-binding fluorescent ligand or salt thereof as claimed in claim 12 selected from M₃ type ligands
N-{2-[4-(2-oxo-2-(6-oxo-5,6-dihydro-11H-pyrido[2,3-b][1,4]benzodiazepine-11-yl)ethyl)-piperazine-1-yl]ethyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{4-[4-(2-oxo-2-(6-oxo-5,6-dihydro-11H-pyrido[2,3-b][1,4]benzodiazepine-11-yl)ethyl)-piperazine-1-yl]butyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{8-[4-(2-oxo-2-(6-oxo-5,6-dihydro-11H-pyrido[2,3-b][1,4]benzodiazepine-11-yl)ethyl)-piperazine-1-yl]octyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{2-[2-(2-(4-(2-oxo-2-(6-oxo-5,6-dihydro-11H-pyrido[2,3-b][1,4]benzodiazepine-11-yl)ethyl)-piperazine-1-yl)ethoxy)ethoxy]ethyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
D1-type ligands
N-{3-oxo-3-[4-(8-methoxy-3-methyl-2,3,4,5-tetrahydro-1H-3-benzazepine-1-yl)phenylamino]propyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{5-oxo-5-[4-(8-methoxy-3-methyl-2,3,4,5-tetrahydro-1H-3-benzazepine-1-yl)phenylamino]pentyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{6-oxo-6-[4-(8-methoxy-3-methyl-2,3,4,5-tetrahydro-1H-3-benzazepine-1-yl)phenylamino]hexyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{8-oxo-8-[4-(8-methoxy-3-methyl-2,3,4,5-tetrahydro-1H-3-benzazepine-1-yl)phenylamino]octyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{4-aza-3,7-dioxo-7-[4-(8-methoxy-3-methyl-2,3,4,5-tetrahydro-1H-3-benzazepine-1-yl)phenylamino]heptyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{4-aza-3,7-dioxo-7-[4-(7-bromo-8-methoxy-3-methyl-2,3,4,5-tetrahydro-1H-3-benzazepine-1-yl)phenylamino]heptyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
mGluR5-type ligands
N-{3-aza-4-oxo-4-[2-chloro-6-(3-chlorobenzyloxy)pyridine-4-yl]butyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}-hexanamide
N-{5-aza-6-oxo-6-[2-chloro-6-(3-chlorobenzyloxy)pyridine-4-yl]hexyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}-hexanamide
N-{9-aza-10-oxo-10-[2-chloro-6-(3-chlorobenzyloxy)pyridine-4-yl]decyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}-hexanamide
N-{9-aza-3,6-dioxa-10-oxo-10-[2-chloro-6-(3-chlorobenzyloxy)pyridine-4-yl]decyl}-6-{2-[4-(2-(4,4-difluoro-4,4a-dihydro-5-(thiophen-2-yl)-4-bora-3a,4a-diaza-s-indacene-3-yl)vinyl)phenoxy]-acetamido}hexanamide
N-{4,7-diaza-3,8-dioxo-8-[2-chloro-6-(3-chlorobenzyloxy)pyridine-4-yl]octyl}-6-{2-[4-(2-(4,4-difluoro-4,4a-dihydro-5-(thiophen-2-yl)-4-bora-3a,4a-diaza-s-indacene-3-yl)vinyl)phenoxy]-acetamido}hexanamide
N-{4,9-diaza-3,10-dioxo-10-[2-chloro-6-(3-chlorobenzyloxy)pyridine-4-yl]decyl}-6-{2-[4-(2-(4,4-difluoro-4,4a-dihydro-5-(thiophen-2-yl)-4-bora-3a,4a-diaza-s-indacene-3-yl)vinyl)phenoxy]-acetamido}hexanamide
N-{6,9-diaza-5,10-dioxo-10-[2-chloro-6-(3-chlorobenzyloxy)pyridine-4-yl]decyl}-6-{2-[4-(2-(4,4-difluoro-4,4a-dihydro-5-(thiophen-2-yl)-4-bora-3a,4a-diaza-s-indacene-3-yl)vinyl)phenoxy]-acetamido}hexanamide
CB1-type ligands
N-{3-aza-4-oxo-4-[5-(4-chlorophenyl)-1-(2,4-dichlorophenyl)-4-methyl-1H-pyrazole-3-yl]butyl}-6-{2-[4-(2-(4,4-difluoro-4,4a-dihydro-5-(thiophen-2-yl)-4-bora-3a,4a-diaza-s-indacene-3-yl)vinyl)-phenoxy]acetamido}hexanamide
N-{5-aza-6-oxo-6-[5-(4-chlorophenyl)-1-(2,4-dichlorophenyl)-4-methyl-1H-pyrazole-3-yl]hexyl}-6-{2-[4-(2-(4,4-difluoro-4,4a-dihydro-5-(thiophen-2-yl)-4-bora-3a,4a-diaza-s-indacene-3-yl)vinyl)-phenoxy]acetamido}hexanamide
N-{9-aza-10-oxo-10-[5-(4-chlorophenyl)-1-(2,4-dichlorophenyl)-4-methyl-1H-pyrazole-3-yl]-decyl}-6-{2-[4-(2-(4,4-difluoro-4,4a-dihydro-5-(thiophen-2-yl)-4-bora-3a,4a-diaza-s-indacene-3-yl)vinyl)-phenoxy]acetamido}hexanamide
N-{2-[4-(1-oxo-1-(5-(4-chlorophenyl)-1-(2,4-dichlorophenyl)-4-methyl-1H-pyrazole-3-yl)methyl)-piperazine-1-yl]ethyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{4-[4-(1-oxo-1-(5-(4-chlorophenyl)-1-(2,4-dichlorophenyl)-4-methyl-1H-pyrazole-3-yl)methyl)-piperazine-1-yl]butyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{8-[4-(1-oxo-1-(5-(4-chlorophenyl)-1-(2,4-dichlorophenyl)-4-methyl-1H-pyrazole-3-yl)methyl)-piperazine-1-yl]octyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{9-aza-3,6-dioxa-10-oxo-10-[5-(4-chlorophenyl)-1-(2,4-dichlorophenyl)-4-methyl-1H-pyrazole-3-yl]decyl}-6-{2-[4-(2-(4,4-difluoro-4,4a-dihydro-5-(thiophen-2-yl)-4-bora-3a,4a-diaza-s-indacene-3-yl)vinyl)-phenoxy]acetamido}hexanamide
N-{3,6-dioxa-8-[4-(1-oxo-1-(5-(4-chlorophenyl)-1-(2,4-dichlorophenyl)-4-methyl-1H-pyrazole-3-yl)methyl)piperazine-1-yl]octyl}-6-{2-[4-(2-(4,4-difluoro-4,4a-dihydro-5-(thiophen-2-yl)-4-bora-3a,4a-diaza-s-indacene-3-yl)vinyl)phenoxy]acetamido}hexanamide
N-{4,9-diaza-3,10-dioxo-10-[5-(4-chlorophenyl)-1-(2,4-dichlorophenyl)-4-methyl-1H-pyrazole-3-yl]decyl}-6-{2-[4-(2-(4,4-difluoro-4,4a-dihydro-5-(thiophen-2-yl)-4-bora-3a,4a-diaza-s-indacene-3-yl)vinyl)-phenoxy]acetamido}hexanamide.

16. A method as claimed in claim 1 wherein the clonal cell line or a ligand identified thereby is suitable for use in the screening of compound libraries for new therapeutic agents, or in the study of pharmacology and signalling characteristics of cell surface receptors.

17.-18. (canceled)

19. The use in the method of claim 1 of a long-lasting, reversibly-binding fluorescent ligand or salt thereof as a reversible fluorescent tag for analysis and sorting, and identifying cells expressing a particular level of receptor at the single cell level in a population expressing such receptors, and for which it is desired to ascertain the level of expression, whereby the specific binding of the fluorescent ligand is an indicator of the level of expression by individual cells in the cell population of the cell surface receptor.

20. (canceled)

21. A method as claimed in claim 1 using long-acting fluorescent ligands or salts thereof that bind to cell surface GPCRs to provide a reversible fluorescent tag for analysis and sorting and identifying in cells expressing a particular level of GPCR at the single cell level, and separating for further expansion in cell culture whereby the fluorescent ligand dissociates from the cells and the clonal cell line produced is the wild type receptor.

22. A method as claimed in claim 1 wherein a cell population is a population of naturally occurring cells and a cell line is a wild type cell line which is cloned from a naturally occurring cell.

23. A method as claimed in claim 1 wherein a cell population comprises cells transfected with a plasmid encoding the known cDNA sequence of the target cell surface receptor and expressing the target cell surface receptor, and the cell line is a recombinant clonal cell line.

24. A method of therapy using a cell line obtained by the method of claim 1.

25. A method as claimed in claim 1, wherein the target cell surface receptor is selected from cannabinoid, metabotropic glutamate, dopamine and muscarinic acetylcholine receptors, and a fluorescent ligand is of the formula:
Lig₃J_{L}L₃J_{Fl}Fl
Or a salt thereof
Wherein Fl comprises a fluorescent moiety and J_{Fl} and J_{L} comprise linking site or linking functionality and
Lig₃ is a non-peptide ligand moiety specific to the target cell surface receptor of formula —X(Z)Ar₁(Y—Ar₂)ₐ
where
X is selected from C, CH or N
Z is selected from H, ═O, C₁₋₆ alk(ox)yl or a single bond linking X and Ar₂
Y is selected from C₁₋₆ alkoxyl such as —OCH₂—, a single bond, C₁₋₆ alkyl, C₁₋₆ amine, C₁₋₆ carbonyl, —NHC(═O)— and a 5-7 membered N-containing saturated heterocycle containing 1, 2 or 3 N atoms
a is a whole number integer 1 or 2
Ar₁ is a 5 or 6 membered (hetero)aromatic, wherein a heteroatom is N, optionally substituted by C₁₋₈ hydrocarbyl, halo, OH and the like, more preferably is selected from the following structures: 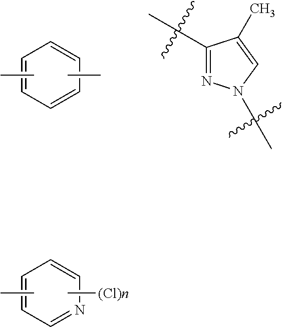
where n is selected from 0 and 1;
and Ar₂ is a 6 membered aromatic optionally substituted by C₁₋₈ hydrocarbyl, halo, OH and the like, more preferably is selected from the following structures: 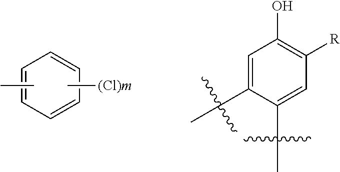
where m is selected from 0, 1 and 2 and R is C₁₋₈ hydrocarbyl, halo, OH and the like;
and L is L₃ and selected from C₁₋₁₂ alkyl, (C₁₋₆ alkoxy)₁₋₅₀C₁₋₁₂alkyl, amide and polyamide moieties including (C₁₋₁₂ alkylNHCOC₁₋₁₂alkyl)₁₋₅₀, (C₁₋₁₂alkylCONHC₁₋₁₂alkyl)₁₋₅₀, (COC₁₋₁₂alkylNH)₁₋₅₀, (NHC₁₋₁₂alkylCO)₁₋₅₀, 5-7 ring heterocyclyl C₁₋₁₂ alk(ox)yl, C₁₋₁₂ alk(ox)yl-5-7 ring heterocyclyl, and C₁₋₁₂ alk(ox)yl 5-7 ring heterocyclyl C₁₋₁₂ alkyl, wherein 1 to 3 heteroatoms, preferably 1 or 2 heteroatoms are selected from N, O and S.

26. A method as claimed in claim 1, wherein the fluorescent ligand is used to both monitor the homogeneity of a cell population expressing a particular cell surface receptor and also to produce the fluorescent signal for cytometry-based cell sorting.
